(19) United States
(12) Patent Application Publication
(10) Pub. No.: US 2011/0309268 A1
(43) Pub. Date: Dec. 22, 2011

(54) ULTRAVIOLET LIGHT BRUSH

(76) Inventor: BLAKE A. PARKER, Auburn, AL (US)

(21) Appl. No.: 13/165,667

(22) Filed: Jun. 21, 2011

Related U.S. Application Data
(60) Provisional application No. 61/356,846, filed on Jun. 21, 2010.

(51) Int. Cl. G21K 5/00

(52) U.S. Cl. 250/492.1

(57) ABSTRACT

The present invention relates to an ultraviolet light brush. The brush has an ultraviolet lamp and a power supply contained within a chamber of the brush. Upon energizing the ultraviolet lamp, ultraviolet light can be emitted from the chamber through a wall and/or the bristles of the brush. The emitted ultraviolet light breaks down at least a portion of the bacteria and/or other microorganisms existing on the brush.

### FIELD OF THE INVENTION

This invention relates generally to the field of cleaning brushes, and more specifically, a brush comprising an ultraviolet light configured to sterilize at least a portion of the microorganisms existing on the brush.

### BACKGROUND OF THE INVENTION

Ultraviolet (UV) sterilization is a sterilization method that uses ultraviolet light at sufficiently short wavelengths to break down microorganisms. UV sterilization is a widely used technology commonly used for air and water sterilization, food and beverage protection, and medical packaging. UV sterilization can also be found in toothbrush and other brush holders.

As part of the light spectrum, ultraviolet light is electromagnetic radiation having a wavelength in the range of between about 10 and 400 nanometers (nm), just below the violet end of the visible spectrum. UV sterilization is a non-chemical approach to disinfection. As such, no chemicals are necessary for disinfection of an item to be cleaned, which makes this process simple, inexpensive and relatively low maintenance. UV sterilizers typically use germicidal lamps configured to produce a certain amount of ultraviolet light.

Brushes used for cleaning perform a multitude of cleaning tasks. For example, there are brushes for cleaning in dry or semi-dry environments, such as brushes used for dusting, sweeping floors, and cleaning shoes. There are also many types of brushes designed for cleaning in wet environments, such as, for example, toothbrushes, dish brushes, toilet brushes, tile brushes, mops and the like. However, these brushes provide an ideal environment for harmful bacteria and viruses to thrive. Thus, what is needed is a brush having sterilizing means so that the brush can remain free of harmful microorganisms.

### SUMMARY

This application relates to systems and methods for the cleaning of brushes, and more specifically, a brush comprising an ultraviolet light configured to sterilize at least a portion of the microorganisms existing on the brush. In one aspect, the UV brush for sterilizing at least a portion of the brush comprises a body, a plurality of bristles attached to the brush, and at least one ultraviolet light source contained within the body. In another aspect, the body can have a handle, a neck, and a head, and can comprise an outer wall defining at least one interior chamber. In another aspect, the plurality of bristles can be formed from an ultraviolet light transmissive material. Each bristle of the plurality of bristles can have a proximal end extending through the outer wall into the at least one interior chamber of the body, and a distal end extending away from the brush. The UV light source can comprise a UV lamp and a power source, and wherein when the lamp is energized, UV light can be transmitted from the proximal end of each bristle of the plurality of bristles to the distal end of the respective bristle.

In another aspect, the UV light brush comprises a body having a handle and a neck and a head selectively coupled to the neck of the brush, wherein at least a portion of the head and/or the bristles can be formed from an ultraviolet light transmissive material. In this aspect, if the head is formed from a UV light transmissive material, the proximal end of each bristle of the plurality of bristles can extend at least partially into the ultraviolet light transmissive material, and the distal end can extend away from the head of the brush. When the UV lamp is energized, UV light can be transmitted through the ultraviolet light transmissive material of the head to the bristles of the brush.

In another aspect, the UV light brush further comprises a brush base, wherein a portion of the ultraviolet light brush is attachable to the brush base. In one aspect, the power source of the brush can be recharged when the ultraviolet light brush is attached to the brush base. In another aspect, the UV lamp can be energized when the UV brush is in use and/or when the UV brush is attached to the brush base.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a perspective view of a UV brush, according to one aspect.

FIG. 2 is a perspective view of a UV brush, according to another aspect.

FIG. 3 is a cross-sectional view of the UV brush of FIG. 3.

FIG. 4 is a side view of base for the brush of FIG. 1 and/or FIG. 2, according to one aspect.

FIG. 5 is a perspective view of a UV brush, according to one aspect.

FIG. 6 illustrates side, partial cross-sectional views of a UV brush, according to one aspect.

### DETAILED DESCRIPTION OF THE INVENTION

The present invention may be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

As used in the specification and the appended claims, the singular forms “a,” “an” and “the” include plural referents unless the context clearly dictates otherwise. Thus, for example, reference to a “bristle” can include two or more such bristles unless the context indicates otherwise.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

As used herein, the term “microorganism” includes for example and without limitation, bacteria, fungi, archaea, protists and viruses.

Reference will now be made in detail to the present preferred embodiment(s) of the invention, examples of which are illustrated in the accompanying drawings. Wherever possible, the same reference numbers are used throughout the drawings to refer to the same or like parts.

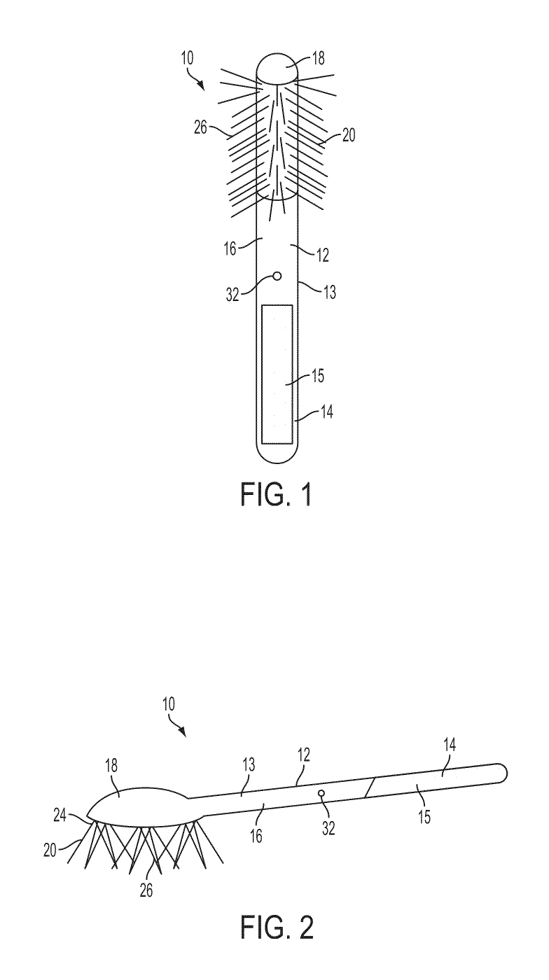

According to various embodiments, a UV light brush 10 is provided as illustrated in FIGS. 1 and 2. In one aspect, the brush can comprise a body 12 having a handle 14, a neck 16 and a head 18. In another aspect, the UV light brush 10 can further comprise a cleaning surface attached to the head of the brush and at least one UV light source contained within at least a portion of the body of the brush. In still another aspect, the cleaning surface can comprise a plurality of bristles 20.

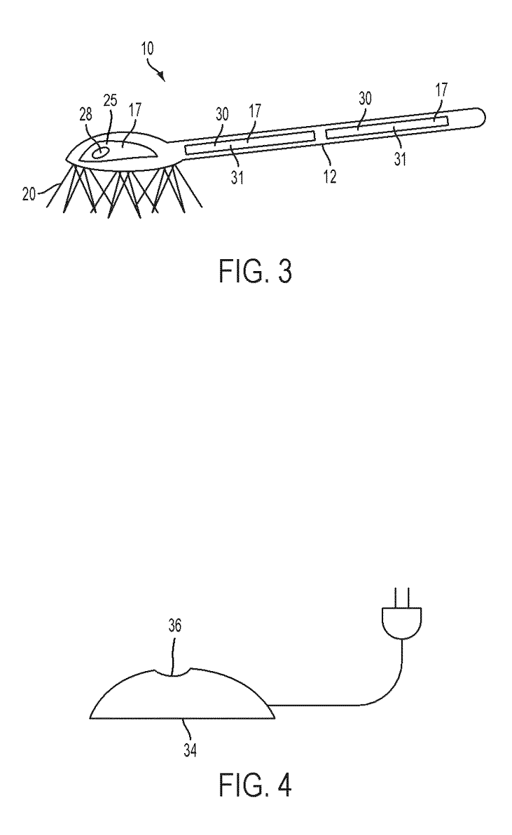

The body 12 of the UV light brush 10 can appear to be a conventional brush body. In one aspect, the handle 14 of the brush can comprise a gripping surface 15 configured to allow a user to more easily grip the brush. In another aspect, portions of the body can be formed from polymeric materials, metallic materials, wooden materials, or any combination thereof. In another aspect, at least a portion of the body 12 can be hollow and can comprise an outer wall 13 shaped so that at least one interior chamber 17 is defined within at least a portion of the body, such as exemplarily illustrated in FIG. 3. For example, in one aspect, a first chamber can be defined within the handle 14, a second chamber can be defined within the neck 16, and a third chamber can be defined within the head 18. However, in another example, a single chamber can be defined within the handle, neck and/or head. In another example, a body chamber can be defined within the handle and/or neck of the brush 10, and a head interior chamber 25 can be defined within the head of the brush. In still another aspect, the at least one interior chamber 17 can be formed from a reflective material. In this aspect, the reflective material of the at least one interior chamber can be configured to reflect UV light in a desired direction (i.e., towards the plurality of bristles 20).

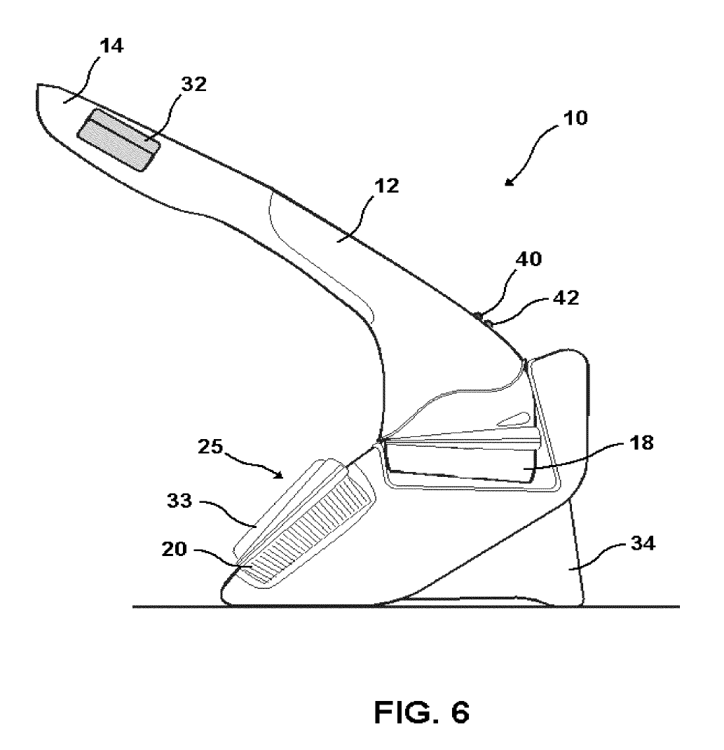

In one aspect, as illustrated in FIGS. 1 and 2, the head 18 of the UV light brush 10 can be formed integrally with the brush. As illustrated in FIG. 6, the head can be configured to be coupled to the body 12 of the brush. In another aspect, the head of the brush can be selectively coupled to the UV light brush. In yet another aspect, the head 18 can be coupled to the brush with a friction fit, a threaded fit, and the like. In this aspect, the head 18 can be detached from the brush 10 for head cleaning and/or replacement. For example, the head 18 can be detached from the brush for head cleaning and a second head 33 can be coupled to the brush so that the brush can continue to be used.

In another aspect, at least a portion of the head 18 of the brush 10 can be formed of a material capable of transmitting UV light through it, such as, for example and without limitation, a polymeric material such as nylon, Plexiglas, and acrylic. In this aspect, at least a portion of the head can be shaped to form at least one lens capable of focusing and/or directing light. For example, at least a portion of the outer wall 13 of the head of the brush can be arcuate in shape so that UV light transmitted through the head can be directed as desired (i.e., towards the bristles 20). In another example, a plurality of arcuate sections can be defined in the head of the brush, wherein each arcuate section can be configured to focus and/or direct light as desired.

In one aspect, each bristle 20 of the plurality of bristles can have a proximal end 24 and a distal end 26. In another aspect, each bristle of the plurality of bristles can be fixedly attached to the head 18 of the brush 10. In one example, the proximal end of each bristle can extend through the outer wall 13 and into the chamber 17 of the head 18 of the brush. In another example, when at least a portion of the head 18 of the brush 10 is formed from a UV light transmissive material, the proximal end 24 of each bristle can be extend at least partially into the UV light transmissive material. In another aspect, the distal end 26 of each bristle 20 can extend away from the head of the brush a predetermined distance. In one aspect, the predetermined distance can be substantially the same for each bristle of the plurality of bristles. In another aspect, the predetermined distance can be different for one or all of the bristles 20. In still another aspect, the bristles can extend from one side of the head, as illustrated in FIGS. 2 and 6. Alternatively, in other aspects, the bristles can extend from all sides of the head 18, as illustrated in FIG. 1, or any portion thereof the head. In still another aspect, each bristle of the plurality of bristles can be substantially linear, curved, or any combination thereof.

In one aspect, some or all of the bristles 20 can be formed from a material capable of transmitting light, such as, for example and without limitation, a polymeric material like nylon, Plexiglas, and acrylic. In another aspect, the bristles can be formed so that they are semi-rigid, that is, the bristles can have a defined shape that can be deformed during the cleaning process so that there is contact between a plurality of bristles and the item to be cleaned. After being deformed, the bristles can at least partly return to their original shape.

In one aspect, the UV light source can comprise a UV lamp 28, a power supply 30, and an on/off button or switch 32, as illustrated in FIGS. 2 and 3. In another aspect, the UV lamp can be a conventional UV lamp that emits light having a wavelength capable of sterilizing an item by breaking down or killing at least a portion of the microorganisms that exist on the item. The UV lamp can be, for example and without limitation, a low-pressure mercury-vapor lamp, configured to emit UV electromagnetic radiation at a desired frequency to kill bacteria, germs, viruses, and/or other microorganisms. In another aspect, at least a portion of the UV lamp 28 can be positioned in the chamber 17 in the head 18 of the brush. In still another aspect, if the head is selectively detachable from the brush, the UV lamp can be positioned in the chamber in the neck of the brush. As will be described more fully below, when the lamp is energized, UV light can be emitted from the lamp 28 in order to contact microorganisms existing on the brush. In still another aspect, the UV light source can comprise a plurality of UV lamps.

In one aspect, the power supply 30 can comprise a conventional power supply, such as a battery compartment and at least one battery 31. In one aspect, the at least one battery can be a rechargeable battery. In another aspect, at least a portion of the power supply can be positioned in the at least one chamber 17 of the brush 10. For example, the battery compartment can be positioned in the interior chamber of the handle 14 and/or neck 16 of the brush, and the at least one battery can be positioned in the battery compartment to power the UV lamp 28. In another aspect, the power supply can comprise a conventional cord and plug configured to connect the UV brush to a conventional household power outlet.

According to another aspect, the on/off button or switch 32 can be a conventional button or switch configured to selectively toggle the flow of power from the power supply 30 to the UV lamp 28, as a user chooses.

With reference to FIG. 6, in one aspect, the UV light brush 10 further comprises an indicator light 40 configured to signal to a user that the UV lamp 28 is energized. For example, the indicator light can be energized by the at least one battery 31 when the UV lamp is energized as a warning to a user that UV light is being emitted by the brush.

In another aspect, the UV light brush 10 further comprises a level light 42 configured to signal to a user the power level of the at least one battery 31. For example, the level light can be energized when the power level of the at least one battery is below a predetermined level so that the user knows the battery must soon be replaced or recharged.

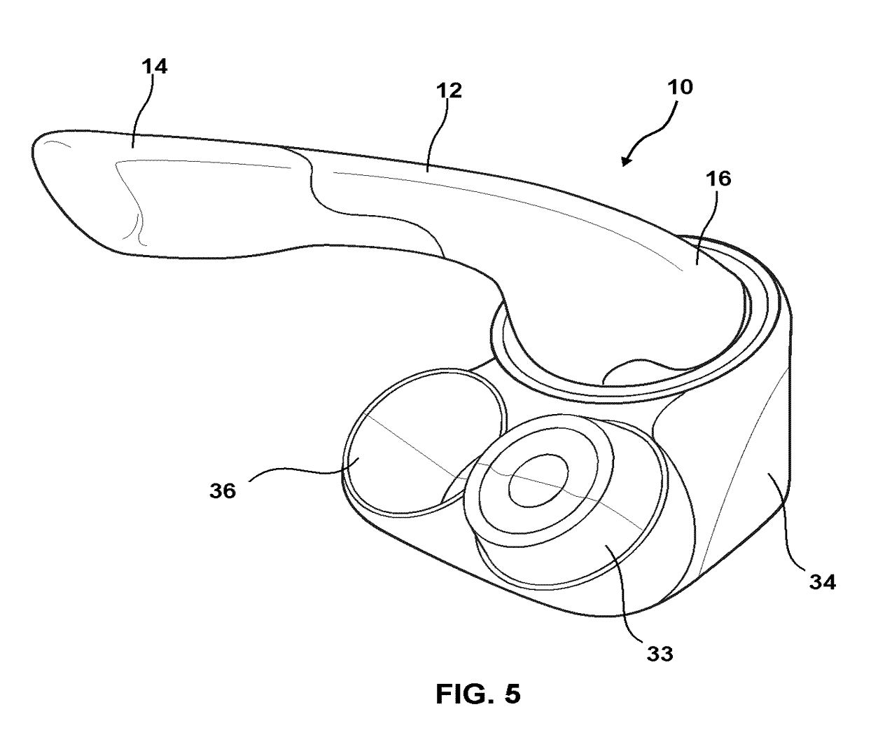

As illustrated in FIGS. 4-6, in one aspect, the UV light brush 10 can further comprise a brush base 34 defining at least one cavity 36. In one aspect, the at least one cavity can be formed from a reflective material such that, in use, UV light emitted by the brush 10 can be reflected from the at least one cavity 36 back towards the brush. In one aspect, the base can be configured to rest on a horizontal surface, such as a table top. Optionally, the base can be attachable to a wall surface. The UV light brush can be attachable to the brush base by inserting a portion of the brush into at least one cavity 36 of the brush base. With reference to FIGS. 5 and 6, for example, the head 18 of the brush can be inserted into the at least one cavity of the brush base 34.

The brush base 34 can be configured to charge the at least one battery 31 of the brush when the brush is placed in the at least one cavity 36 of the brush base. In one aspect, the at least one battery 31 of the brush can be charged by inductive charging. In another aspect, the UV lamp 28 can be energized when the brush 10 is placed in the brush base. In another aspect, the UV lamp can automatically be energized for the entire time that the brush is placed in the brush base, or alternatively, the UV lamp can automatically be energized for a predetermined period of time when the brush 10 is placed in the base 34. For example, when the brush 10 is placed in the brush base, the UV lamp 28 can be energized for about 10 seconds, 20 seconds, 30 seconds, 40 seconds, 50 seconds, 1 minute, 2 minutes, 3 minutes, 4 minutes, 5 minutes, 6 minutes, 7 minutes, 8 minutes, 9 minutes, 10 minutes, 15 minutes, 20 minutes, 30 minutes, 45 minutes, 1 hour, 1.5 hours, 2 hours, 3 hours, 4 hours, 5 hours, 6 hours, 7 hours, 8 hours, 10 hours, 12 hours, 14 hours, 16 hours, 18 hours, 20 hours, 22 hours, or 24 hours. In another aspect, the brush base 34 can emit a scent when the UV lamp 28 is energized. It is contemplated that the scent can give a user the impression that the brush is being cleaned.

In one aspect, if the head 18 is selectively detachable from the UV light brush 10, the UV brush can further comprise a plurality of heads. For example, a first head 18 of the plurality of heads can be attached to the brush and a second head 33 can be attached to the brush base 34. In this manner, the second head can be cleaned in the base while the first head is in use. Alternatively, the first head can be discarded if desired by the user (because of wear, etc.) and the brush can continue to be used with the second head. Optionally, the second head 33 can be sized and/or shaped differently from the first head so that the head can be used for cleaning different items. For example, the first head could be sized and configured for cleaning dishes, and the second head could be sized and configured for cleaning floors.

In use, the brush 10 can be used to clean an item as a conventional brush would be used. For example and without limitation, the brush can be a dish brush for cleaning dishes, a scrubbing brush for cleaning cutting boards, a toothbrush for cleaning teeth, toilet brush for cleaning toilets, and a wet mop for cleaning a floors. If the brush is a wet mop, for example, the UV lamp 28 can be positioned in the head of the mop, the power supply can be positioned in the handle of the mop, and UV light can be emitted from the head of the mop to the surface being cleaned and/or to the bristles, pad, yarn, or other cleaning surface of the mop. It is of course contemplated that the brush can be at least partially water resistant, so that exposing the brush to water and other liquids will not damage the power supply 30 contained within the chamber 17 of the brush.

In one aspect, after use and/or during use of the brush 10, energy from the power supply can energize the UV lamp 28. For example, a user can toggle the on/off button or switch 32 to the “on” position, or the lamp can become automatically energized upon placing the brush in the brush base 34. In one aspect, at least a portion of the UV light emitted from the UV lamp can be received by the respective bristles at the proximal end 24 of the bristles. It is contemplated that at least a portion of this UV light can communicate or travel down the bristle toward the distal end 26 of each bristle so that at least a portion of the bacteria and/or other microorganisms existing on the brush are exposed to a desired and beneficial level of the sterilizing UV light. In another aspect, if at least a portion of the head 18 of the brush 10 is formed from a light transmissive material, at least a portion of the UV light emitted from the UV lamp 28 can be transmitted through the light transmissive material of the head so that at least a portion of the microorganisms existing on the brush are exposed to a desired and beneficial level of the sterilizing UV light. In still another aspect, it is contemplated that UV light can be transmitted through the light transmissive material of the head and/or the bristles 20 of the brush in order to expose at least a portion of the microorganisms existing on the brush to a desired and beneficial level of the sterilizing UV light. One will appreciate that, upon exposure to the UV light, the microorganisms can be broken down (killed) so that portions of the brush 10 without any microorganisms existing thereon are sterilized.

Further, at least a portion of the microorganisms existing on the item being cleaned by the UV brush 10 can be exposed to the UV light emitted from the brush. For example, if the brush was used to clean a cutting board, UV light emitted from the brush 10 can kill at least a portion of the microorganisms existing on the cutting board. In another example, if the brush is a mop used for cleaning a floor, UV light emitted from the mop can kill at least a portion of the microorganisms existing on the floor.

In another aspect, after use of the brush 10, the user can place the brush on the brush base 34. In one aspect, on the base, the UV lamp can become automatically energized, and UV light can be emitted from the UV lamp to the microorganisms existing on the brush. It is contemplated that the time and frequency of the emission of the UV light can be selected by the user and/or can be preset at the manufacturing facility.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is therefore understood that the invention is not limited to the specific embodiments disclosed herein, and that many modifications and other embodiments of the invention are intended to be included within the scope of the invention. Moreover, although specific terms are employed herein, they are used only in a generic and descriptive sense, and not for the purposes of limiting the described invention.

What is claimed is:

1. An ultraviolet light brush for sterilizing at least a portion of the brush comprising:
a body having a handle, a neck, and a head, wherein the body comprises an outer wall defining at least one interior chamber;
a plurality of bristles attached to the head of the brush and formed from an ultraviolet light transmissive material, wherein each bristle of the plurality of bristles has a proximal end extending through the outer wall into the at least one interior chamber, and a distal end extending away from the head of the brush; and
at least one ultraviolet light source contained within the at least one interior chamber of the brush for producing ultraviolet light, wherein the ultraviolet lamp emits light having a wavelength capable of sterilizing at least a portion of the brush, and wherein ultraviolet light emitted from the ultraviolet light source is transmitted from the proximal end of each bristle of the plurality of bristles to the distal end of the respective bristle.

2. The ultraviolet light brush of claim 1, wherein the at least one ultraviolet light source comprises an ultraviolet lamp and means for energizing the ultraviolet lamp.

3. The ultraviolet light brush of claim 2, wherein the at least one interior chamber comprises a head interior chamber defined in the head of the brush, and wherein the ultraviolet lamp is positioned in the head interior chamber.

4. The ultraviolet light brush of claim 2, wherein the means for energizing the ultraviolet lamp comprises at least one battery.

5. The ultraviolet light brush of claim 4, wherein the at least one battery is a rechargeable battery.

6. The ultraviolet light brush of claim 5, wherein the ultraviolet light brush further comprises a brush base, wherein a portion of the ultraviolet light brush is attachable to the brush base, and wherein the at least one battery is at least partially recharged when the ultraviolet light brush is attached to the brush base.

7. The ultraviolet light brush of claim 6, wherein the ultraviolet lamp is energized when the ultraviolet brush is attached to the brush base.

8. The ultraviolet light brush of claim 7, wherein the ultraviolet lamp is energized for a predetermined period of time when the ultraviolet brush is attached to the brush base.

9. The ultraviolet light brush of claim 8, wherein the ultraviolet light brush further comprises an indicator light configured to signal to a user that the ultraviolet lamp is energized.

10. The ultraviolet light brush of claim 8, wherein the ultraviolet light brush further comprises an indicator light configured to signal to a user the power level of the at least one battery.

11. The ultraviolet light brush of claim 1, wherein at least a portion of the outer wall of the head of the brush is formed from an ultraviolet light transmissive material.

12. An ultraviolet brush system comprising:
an ultraviolet light brush for sterilizing at least a portion of the brush comprising:
a body having a handle and a neck, wherein the body comprises an outer wall defining at least one body interior chamber;
a head selectively coupled to the neck of the brush comprising an outer wall defining at least one head interior chamber;
a plurality of bristles attached to the head of the brush and formed from an ultraviolet light transmissive material, wherein each bristle of the plurality of bristles has a proximal end extending through the outer wall into the at least one head interior chamber, and a distal end extending away from the head of the brush; and
at least one ultraviolet light source comprising at least one ultraviolet lamp and at least one battery, the at least one ultraviolet lamp and at least one battery contained within the at least one body interior chamber of the brush, wherein the ultraviolet lamp emits light having a wavelength capable of sterilizing at least a portion of the brush, and wherein ultraviolet light from the ultraviolet lamp is transmitted from the proximal end of each bristle of the plurality of bristles to the distal end of the respective bristle; and
a brush base, wherein a portion of the ultraviolet light brush is attachable to the brush base, and wherein the at least one battery is at least partially recharged when the ultraviolet light brush is attached to the brush base.

13. The ultraviolet brush system of claim 12, wherein the head is selectively coupled to the neck of the brush.

14. The ultraviolet brush system of claim 13, wherein the ultraviolet lamp is energized when the ultraviolet brush is attached to the brush base.

15. The ultraviolet brush system of claim 13, wherein the brush base defines at least one cavity and wherein a portion of the head of the ultraviolet light brush is attachable to the at least one cavity.

16. The ultraviolet brush system of claim 15, wherein an inner surface of the at least one cavity is formed from a reflective material.

17. The ultraviolet brush system of claim 15, further comprising a second head, and wherein the second head is selectively coupled to the neck of the ultraviolet brush when the first head is attached to the brush base.

18. The ultraviolet brush system of claim 12, wherein the brush base emits a scent when the ultraviolet lamp is energized.

19. An ultraviolet light brush system for sterilizing at least a portion of the brush comprising:
a body having a handle and a neck, wherein the body comprises an outer wall defining at least one interior chamber;
a head selectively coupled to the neck of the brush, wherein at least a portion of the head is formed from an ultraviolet light transmissive material;
a plurality of bristles attached to the head of the brush, wherein each bristle of the plurality of bristles has a proximal end extending at least partially into the ultraviolet light transmissive material, and a distal end extending away from the head of the brush; and
at least one ultraviolet light source comprising at least one ultraviolet lamp positioned in the at least one interior chamber of the brush, and at least one battery positioned in the at least one interior chamber of the brush, wherein the ultraviolet lamp emits light having a wavelength capable of sterilizing at least a portion of the brush, and wherein ultraviolet light from the ultraviolet lamp is transmitted through the ultraviolet light transmissive material of the brush.

20. The ultraviolet light brush of claim 19, further comprising a brush base, wherein a portion of the ultraviolet light brush is attachable to the brush base, and wherein the at least one battery is at least partially recharged when the ultraviolet light brush is attached to the brush base.

21. The ultraviolet light brush of claim 19, wherein the at least one interior chamber is formed from a reflective material.

22. The ultraviolet light brush of claim 19, wherein each bristle of the plurality of bristles is formed from an ultraviolet light transmissive material, and wherein ultraviolet light from the ultraviolet lamp is transmitted from the proximal end of each bristle of the plurality of bristles to the distal end of the respective bristle.
